(19) United States
(12) Patent Application Publication
(10) Pub. No.: US 2010/0049241 A1
(43) Pub. Date: Feb. 25, 2010

(54) RATCHET HOOK TOURNIQUET

(76) Inventor: James L. Persson, Enterprise, AL (US)

Correspondence Address: United States Army Medical Research;and Material Command MCMR-ZA-J, 504 Scott Street, Fort Detrick, MD, 21702-5012, US

(21) Appl. No.: 12/540,139

(22) Filed: Aug. 12, 2009

Related U.S. Application Data
(60) Provisional application No. 61/090,042, filed on Aug. 19, 2008.

(51) Int. Cl. A61B 17/00

(52) U.S. Cl. 606/203

(57) ABSTRACT

A novel tourniquet device is disclosed comprising a belt with a separable first end and second end, preferably coupled by hook-and-pin means, said hook being designed so as to be easily discernable from fabric by touch and easily manipulated manually. Constrictive tension is applied preferably by a ratchet action mechanism amongst other means.

### REFERENCE TO RELATED APPLICATIONS

This application claims the benefit of Provisional Application No. 61/090,042, filed Aug. 19, 2008; the contents of which are hereby incorporated by reference in their entirety.

### RIGHTS IN THE INVENTION

This invention was made with support from the United States Government and, specifically, the United States Army Medical Research & Materiel Command and, accordingly, the United States government has certain rights in this invention.

### FIELD AND BACKGROUND OF THE INVENTION

The invention is an improvement of the traditional ratchet tourniquet used to stop uncontrollable bleeding from gunshot wounds and blast injuries to the arms and legs. Disclosed is a device with a detachable flat metal hook, allowing the loop of the ratchet tourniquet to open up.

Currently, there are two commonly used types of tourniquets: the combat application tourniquet (CAT) (FIGS. 1A and 1B) and the ratchet tourniquet (FIGS. 2A and 2B). The CAT (FIGS. 1A and 1B) consists of a VELCRO™ (Velcro USA Inc., Manchester, N.H.)(aka hook-and-loop fastener) belt attached to a windlass assembly. By keeping the CAT with the belt fed partially through the friction buckle, the tourniquet can be applied by the injured person with one arm. In this one-handed configuration, the loop is fed over the end of the injured extremity, advanced up the injured limb and tightened with the windlass. A medic can also open the CAT and pass the belt under the extremity and fasten the tourniquet higher up on the extremity. This two-handed configuration avoids the need to pass the loop around and up the injured extremity. In this case, the free end of the belt must be fed under the injured extremity with one hand, retrieved by the other hand and then fed through a friction buckle before being tightened.

Although a medic can apply the one-handed configuration to an injured limb, the main disadvantage is the need to feed the loop over the injured limb and advance upward. This process can be quite difficult when passing the loop over disfiguring injuries of the limbs or in cramped places like vehicles or helicopters. While using the two-hand CAT method, feeding the cloth belt under the injured extremity can be difficult in noisy, cluttered or dark environments. In such situations, the belt can become twisted during passage under injured limb. Since the belt is made of cloth, the ability for the retrieving had to differentiate between this cloth belt from the patient's clothing and equipment can be uncertain. Passage of the belt through the friction buckle is both time consuming and cumbersome with bloody hands in extreme situations. Furthermore, the VELCRO™ can be fouled with blood and dirt, lessening its adhesiveness and ability to maintain the loop integrity while tightening. Finally, the windlass assembly can be knocked loose during rough transport.

In order to get a more secure tourniquet placement, medics will often use the traditional ratchet tourniquet (FIGS. 2A and 2B). Similar to the CAT, this device forms a loop that must be fed over the injured limb and advanced upward past the injury before tightening high up on the limb. Unlike the plastic and cloth assembly of the CAT, the metal ratchet mechanism of this tourniquet forms a strong bond that will not dislodge with even the roughest handling. However, similar to the one-handed configuration of the CAT, the main disadvantage is the need to pass the injured limb through the loop.

Therefore, there exists a need for an improved tourniquet device and method of application that allows for the rapid and secure application of the tourniquet not currently available in the art.

### SUMMARY OF THE INVENTION

A tourniquet device comprising: a flexible element with at least a first end and a second end; a fastening element integral with the first end of the flexible element, said fastening element comprising a flat hook; a locking element integral with the second end of the flexible element, said locking element being operative to removably relate to the fastening element; a tension producing mechanism operative to apply tension to said flexible element.

Current ratchet tourniquets incorporate a permanently sewn connection between the belt and the body of the ratchet, forming a loop that has to be advanced up the injured limb before being tightened (see FIGS. 2A and 2B). Since the injured limb must be fed through the loop, precious time is lost during this process, especially while applying a tourniquet in cramped places like vehicles or helicopters. Instead of a sewn connection, the preferred embodiment of the present invention employs a device that utilizes a flat metal hook that connects the belt to the body of the ratchet. This allows the tourniquet to open up and the free end to pass around the injured limb. Rather than passing the injured limb through a tourniquet loop, the opened tourniquet can be applied immediately above the injury and tightened without further disruption of the limb. This modified application method would allow faster and easier application of the tourniquet.

The disclosure describes an improved tourniquet to stop otherwise uncontrollable bleeding from limbs. The tourniquet would be used under conditions of severe extremity trauma, such as gunshot wounds or blast injury. The invention is specifically directed at improving the certainty of effective placement and the speed of application of the tourniquet in cramped environments such as vehicles, and when the limb has suffered severe disfiguring trauma. The preferred embodiment utilizes a detachable hook-and-pin attachment, the hooked end of the tourniquet belt can be manually fed around the limb, and re-attached to the ratchet and tightened, rather than requiring that the tourniquet belt be fed over the end of the limb and advanced to above the wound before tightening as is the case in the standard tourniquet configuration known in the art. Furthermore, the detachable hook configuration allows the belt to be fed cleanly around the limb and attached to the ratchet mechanism with less chance of twisting during application.

This application discloses a device that allows for the application of a tourniquet more rapidly than either of the other types of tourniquets previously discussed. With the CAT (FIGS. 1A and 1B) in the one-handed loop configuration, the injured extremity must be passed through the loop while the tourniquet is advanced up the limb until positioned properly. This action is quite difficult with mutilating injuries of extremities in cramped condition, such as responding to a vehicle hit by a roadside bomb or treating a patient in a helicopter. The two-handed CAT application loses valuable time while the belt is retrieved and fed through the friction buckle. As opposed to feeding a belt through two narrow slots in a friction buckle, the large metal hook of the preferred embodiment of the invention is simply hooked on to the pin in the ratchet.

Likewise, the typical ratchet tourniquet (FIGS. 2A and 2B) is cumbersome due to the need to pass the injured extremity through the loop. Overall, the preferred embodiment of the present invention can be applied within 4-5 seconds with practice. Most importantly, the resulting application is very secure and should not dislodge with further medical evacuation.

The main use for the disclosed tourniquet is in wartime situations where bleeding must be controlled quickly while possibly under enemy fire. In this situation, the tourniquet application must be rapidly with as few steps as possible. The resulting application must also be secure enough to ensure continued bleeding control during rough medical evacuation.

It is an object of the present invention to enable the rapid and secure application of a tourniquet under extreme conditions through the incorporation of a flat metal hook into the belt of a ratchet tourniquet, allowing the loop to open up. This hook is broad to keep the belt from twisting during application and to ensure a stable bond to the ratchet. The broad width of the hook allows the connection to swivel up and down, not side to side, allowing the connection to conform to the body surface. Using a hooking mechanism as opposed to a friction buckle significantly lessens application time and effort.

It is another object of the present invention to improve the certainty of effective placement and the speed of application of the tourniquet in cramped environments such as vehicles, and when the limb has suffered severe disfiguring trauma. By modifying the current fixed pinning of the tourniquet belt to the ratchet tightening mechanism to a detachable hook-and-pin attachment, the hooked end of the tourniquet belt can be manually fed around the limb, and re-attached to the ratchet and tightened, rather than requiring that the tourniquet belt be fed over the end of the limb and advanced to above the wound before tightening as is the case in the current tourniquet configuration.

Yet a further object of the present invention is to provide a device with a detachable hook configuration that allows the belt to be fed cleanly around the limb and attached to the ratchet mechanism with less chance of twisting during application.

The various features of novelty that characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1A is a drawing showing the closed one-handed loop configuration of the Combat Application Tourniquet (CAT):

FIG. 1B is a drawing showing the open two-handed belt configuration of the Combat Application Tourniquet (CAT):

FIG. 2A is a drawing of a traditional ratchet design with sewn junction.

FIG. 2B is a drawing of a magnified view of the traditional ratchet design feature.

FIG. 3A is a drawing of an embodiment of this invention demonstrating a metal hook attachment.

FIG. 3B is a drawing of a magnified view of the metal hook attachment element of the preferred embodiment of the invention.

FIG. 4 is a drawing demonstrating the introduction of hook and belt under injured extremity.

FIG. 5 is a drawing demonstrating the retrieval of hook and advancement of belt by opposite hand.

FIG. 6 is a drawing demonstrating the attachment of metal hook and tourniquet belt to the coupling pin of ratchet-action mechanism.

### DESCRIPTION OF THE PREFERRED EMBODIMENTS

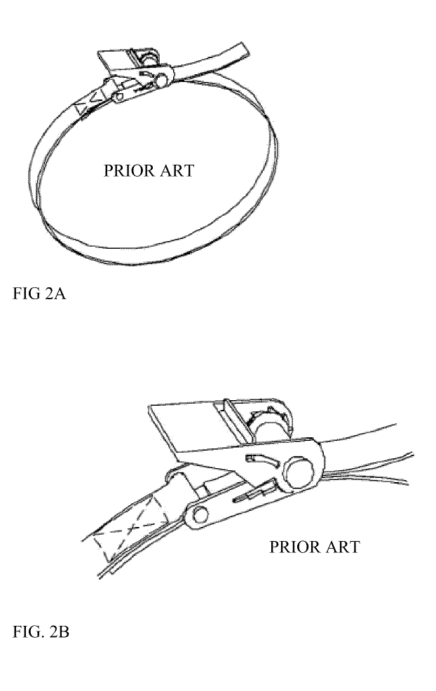

The preferred embodiment is an improvement of the traditional ratchet tourniquet used to stop uncontrollable bleeding from gunshot wounds and blast injuries to the arms and legs. The currently used ratchet tourniquet has one end of the belt fed through a ratchet mechanism while the other end is permanently sewn around a pin in the ratchet body (FIGS. 2A and 2B). In the preferred embodiment of the invention, this permanently attached end of the ratchet belt is replaced with a detachable flat metal hook, allowing the loop of the ratchet tourniquet to open up.

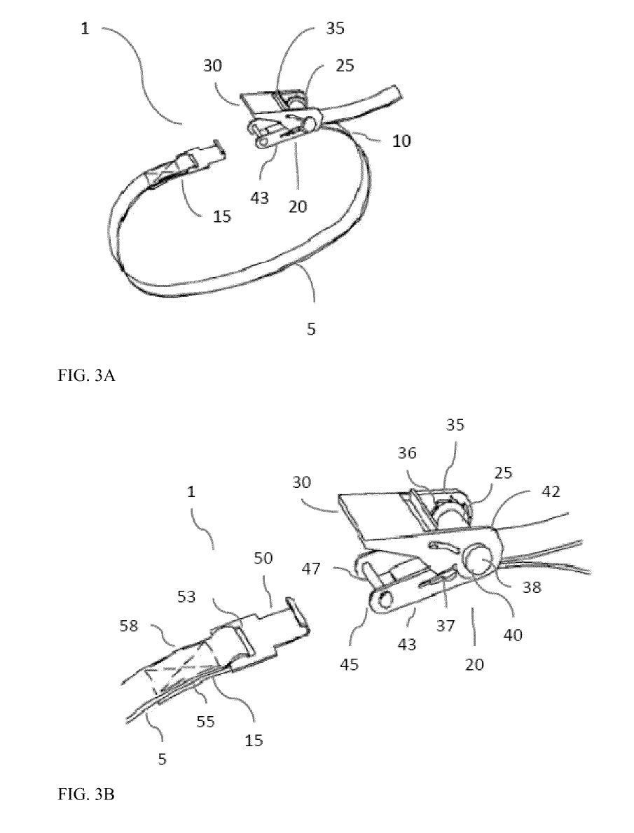

Referring to FIGS. 3A and 3B, the tourniquet device of this invention as illustrated in the various embodiments herein is generally designated as 1. The invention has a belt 5, with a first end 10 and second end 15. The first end 10 of the belt 5 is engaged with a ratchet device, generally designated as 20. The ratchet device 20 preferably includes a cylindrical spool generally designated as 25 to which the first end 10 of the belt 5 is affixed for being spooled, a lever 30, and a ratchet-action mechanism 35 which rotates the spool in a spooling direction whenever a user operates the lever 30. Such a ratchet-action mechanism preferably comprises of ratchet teeth 36 carried by the spool 25 and a spring-pressed holding pawl 37 to prevent reverse rotation of the spool together with a spring-pressed driving pawl (not shown) to rotate the spool in the winding direction. Alternatively, the spring-pressed holding pawl (not shown) may be replaced with a fixed lug means (not shown) to serve the function of a holding pawl and by mounting the spool 25 in a floating manner, the arrangement being such that the tension of the belt 5 urges the spool 25 into engagement with the fixed holding lug (not shown). Thus the belt 5 itself provides the yielding force that would otherwise be provided by a special spring. In the preferred embodiment, the lever 30 is operated by reciprocally rotating the lever 30 around the ratchet mechanism axis 38. By way of nonlimiting example the ratchet mechanism comprises a pintle 40 and gudgeon 42. The ratchet device 20 further includes an elongated arm 43 which extends from the ratchet-action mechanism 35 and has a distal end 45 integrated with a coupling pin 47. The second end 15 of the belt 5 is engaged with a hook 50 designed to clasp with the coupling pin 47 of the elongated arm 43 of the ratchet-action mechanism 35. In the preferred embodiment, the hook 50 further comprises an integral slot 53 through which the second end 15 of the belt is thread. The lead end 55 of the second end 15 is sewed back onto the lagging portion 58 once it has been threaded through the integral slot 53, forming a fixed engagement with the hook 50. In the preferred embodiment, the hook comprises a metal flat hook, also known in the art as a gutter hook, curved hook, or vehicle hook.

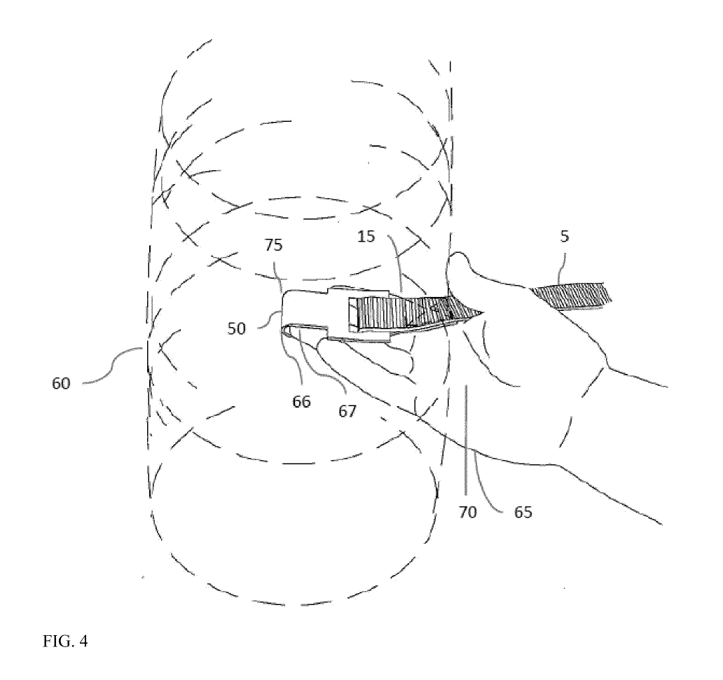

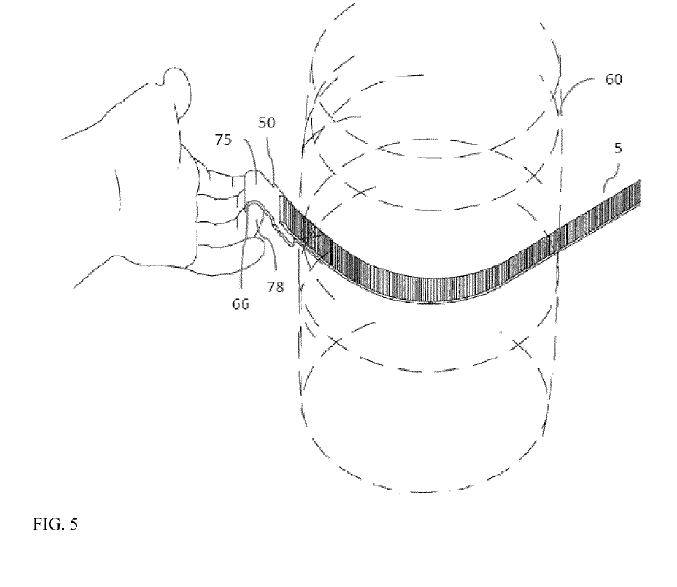

This design allows the tourniquet 1 to be applied at a level above the injury instead of passing the injured limb through the loop of the traditional ratchet tourniquet. Referring to FIGS. 4 and 5, during application, the hook 50 and the second end 15 of the belt 5 are fed under the injured extremity 60 (shown with dashed lines to indicate transparency) with one hand 65. The broad curved portion of hook 66 wraps partially around fingertips 67, providing protection for leading fingers. Meanwhile, the other hand 68 retrieves the hook 50 and advances the belt 5 further from the other side of the injured extremity 60. Only the fingertips of the hand are needed to identify and retrieve the curved portion of hook.

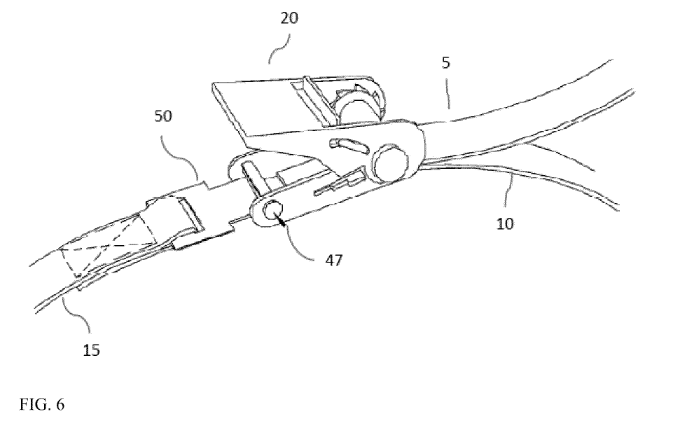

Turning now to FIG. 6, once the hook 50 and second end 15 of the belt 5 is passed around the extremity 60, the hook 50 is fastened into the ratchet-action mechanism 35 by clasping the coupling pin 47. The tourniquet 1 is now cinched down using the ratchet-action mechanism 35 as described herein. The tension in the tightened belt 5 and the outward force from the compressed tissue of the extremity 60 keep the hook 50 securely fastened to the coupling pin 47.

In an alternative embodiment the tension producing mechanism may comprise a windlass as disclosed in U.S. patent application Ser. No. 11/147,806 to Esposito and Ser. No. 11/410,638 to Rutherford and U.S. Pat. No. 6,899,720 to McMillan and (which are incorporated by reference in their entirety herein).

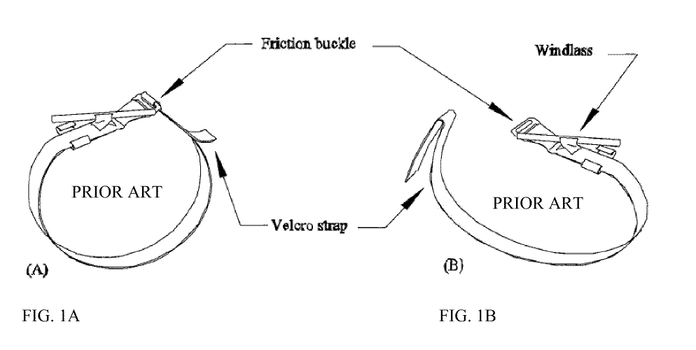

In yet another alternate embodiment, the tension to the belt may be applied using hook-and-loop means as described above with respect to the Combat Application Tourniquet (CAT) (see also, FIGS. 1A and 1B).

In yet a further alternate embodiment, the tension producing mechanism may comprise a slip buckle with frictional bias means as disclosed in U.S. Pat. No. 6,960,223 to Ambach, U.S. Pat. No. 6,217,601 to Chao, U.S. Pat. No. 6,884,254 to Brooks, U.S. Pat. No. 1,447,967 to Davis and U.S. Pat. No. 2,113,534 to Brown (which are incorporated by reference in their entirety herein).

The use of a metal hook 50 at the second end 15 of the belt 5 offers several advantages over the prior art. When the belt 5 is held in the palm of the hand 70, the flat hook curves 50 over the fingertips of the middle and ring fingers 67 (see FIGS. 5 and 6). The flat portion 75 of the hook 50 “locks” the hook against the palm side of the fingers 67, keeping the belt from twisting while passing under the extremity (see FIGS. 5 and 6). Unlike the cloth belt of the CAT, the flat metal hook 50 covers and protects the driving fingertips 67, forming a strong metal interface to separate the injured extremity 60 from dirt, rocks, seat cushions, etc. (not shown) that surrounds the patient (not shown).

With the hook 50 passed to the maximal extent, the fingertips on the retrieving hand 78 easily grasp around the hook 50 and pull the belt 5 around the remainder of the injured extremity 60 (FIG. 5). Unlike the cloth belt of the CAT, the metal hook 50 is easily distinguishable from the surrounding clothes, tissue, and equipment belts (not shown) that may make finding the tip of a cloth belt difficult. Once passed around the extremity, the hook 50 is fastened into the ratchet-action mechanism 35 by clasping it to the coupling pin 47. Fastening the hook to the ratchet is much simpler and quicker than feeding a cloth belt through the friction buckle of the CAT. The tourniquet 1 is now cinched down and tightened just like a regular ratchet tourniquet.

While a specific embodiment of the invention will be shown and described in detail to illustrate the application of the principles of the invention, it will be understood that the invention may be embodied otherwise without departing from such principles.

What is claimed is:

1. A tourniquet device comprising:
a flexible element with at least a first end and a second end;
a fastening element integral with the first end of the flexible element, said fastening element comprising a flat hook;
a locking element integral with the second end of the flexible element, said locking element being operative to removably relate to the fastening element;
a tension producing mechanism operative to apply tension to said flexible element.

2. The device of claim 1 wherein the flexible element comprises a flexible polymeric material.

3. The device of claim 2, wherein the flexible polymeric material comprises a material selected from a group consisting of acetal, nylon, polypropylene, and polyethylene.

4. The device of claim 1, wherein the locking element comprises a pin.

5. The device of claim 1, wherein the tension producing mechanism is a ratchet.

6. The device of claim 1, wherein the tension producing mechanism is a windlass.

7. The device of claim 1, wherein the tension producing mechanism is a slip buckle with frictional bias means.

8. The device of claim 1, wherein the tension producing mechanism is a hook-and-loop fastener.

9. A tourniquet device comprising:
a flexible element with a first end and a second end;
a fastening element integral with the first end of the flexible element, said fastening element comprising a flat hook;
a locking element being operative to removably relate to the fastening element, said locking element forming a first part of an assembly;
a tension producing mechanism that is integral with the locking element, said tension producing mechanism forming a second part of the assembly, said assembly being integral with the second end of the flexible element.

10. The device of claim 9 wherein the flexible element comprises a flexible polymeric material.

11. The device of claim 10, wherein the flexible polymeric material comprises a material selected from a group consisting of acetal, nylon, polypropylene, and polyethylene.

12. The device of claim 9, wherein the locking element comprises a pin.

13. The device of claim 9, wherein the tension producing mechanism is a ratchet.

14. The device of claim 9, wherein the tension producing mechanism is a windlass.

15. The device of claim 9, wherein the tension producing mechanism is a slip buckle with frictional bias means.

16. The device of claim 9, wherein the tension producing mechanism is a hook-and-loop fastener.

17. A method of applying a tourniquet comprising:
providing a constriction device having a belt; a coupler configured to couple a first end of said belt with a second send of said belt, said coupler comprising a flat hook, and a tension producing mechanism to apply tension to said belt;
manipulating the first end of said belt around a body part;
coupling the first end of said belt with the second end of said belt to encircle the body part; and,
adjusting the tension producing mechanism to increase constrictive pressure around the body part sufficient to prevent further loss of blood.

18. The method of claim 17, wherein the coupler further comprises a pin.

19. The method of claim 17, wherein said coupler is integral with said tension producing mechanism.

20. The method of claim 17, wherein said tension producing mechanism comprises a ratchet.
